(19) United States
(12) Patent Application Publication
(10) Pub. No.: US 2024/0376470 A1
(43) Pub. Date: Nov. 14, 2024

(54) PHARMACEUTICAL COMPOSITION FOR PREVENTING OR TREATING COLON CANCER INCLUDING EXTRACELLULAR VESICLES LOADED WITH MACC1 INHIBITOR AS ACTIVE INGREDIENT

(71) Applicant: THE CATHOLIC UNIVERSITY OF KOREA INDUSTRY-ACADEMIC COOPERATION FOUNDATION, Seoul (KR)

(72) Inventors: Byung Jo CHOI, Daejeon (KR); Say June KIM, Seoul (KR); Ok Hee KIM, Chungcheongnam-do (KR)

(21) Appl. No.: 18/659,711

(22) Filed: May 9, 2024

(30) Foreign Application Priority Data
May 9, 2023 (KR) 10-2023-0059825

(51) Int. Cl. C12N 15/113; A61K 9/00; A61K 9/50; A61P 35/00

(52) U.S. Cl. CPC C12N 15/113; A61K 9/0019; A61K 9/5063; A61P 35/00; C12N 2310/141

(57) ABSTRACT

Disclosed is a pharmaceutical composition for preventing or treating colon cancer comprising extracellular vesicles loaded with an MACC1 inhibitor as an active ingredient. The pharmaceutical composition allows the extracellular vesicles loaded with the MACC1 inhibitor, a colon cancer-promoting factor, to deliver the inhibitor specifically to colon cancer cells and is characterized by high stability and biodegradability, thereby having a low risk of side effects and excellent drug delivery. Therefore, the pharmaceutical composition of the present disclosure is expected to be utilized in a variety of ways as a treatment for colon cancer with minimal risk of side effects and maximized anticancer ability.

### CROSS-REFERENCE TO RELATED APPLICATION

This application claims priority to Korean Patent Application No. 10-2023-0059825, filed on May 9, 2023, the disclosure of which is incorporated by reference herein in its entirety.

### REFERENCE TO A “SEQUENCE LISTING,” SUBMITTED AS AN XML FILE

The Sequence Listing written in the xml file titled: “206132-0176-OOUS_SequenceListing.xml”; created on May 7, 2024, and 3,943 bytes in size, is hereby incorporated by reference.

### BACKGROUND OF THE INVENTION

### 1. Field of the Invention

The present disclosure relates to a pharmaceutical composition for preventing or treating colon cancer including extracellular vesicles loaded with an MACC1 inhibitor as an active ingredient.

### 2. Description of the Related Art

Colon cancer mainly occurs in people living in the United States or Europe who eat a lot of animal fat and meat, and is cancer with the second highest incidence and mortality rate, especially in the United States. In Asian countries including Korea and Japan, the incidence rate is lower than in the West, but the incidence rate of colon cancer has been increasing recently as dietary habits have become westernized. The cause of colon cancer has not yet been clearly identified, but familial polyposis, idiopathic non-specific ulcerative colitis, and polyps of the colon and rectum, especially villous adenoma, are known to be diseases that can turn into cancer.

Colon cancer can be treated by removing polyps using an endoscope before it turns into cancer, or in the case of small-sized colon cancer in the form of polyps, it can be treated with endoscopic resection alone. In addition, the main treatment for colon cancer is surgery, and radiation therapy is performed in addition to anticancer drugs after the surgery. However, anticancer drugs and radiation therapy have side effects that cause damage to normal cells as well as cancer cells.

Meanwhile, miRNA, which is a small non-coding RNA consisting of 18-25 nucleotides (nt), regulates gene expression by binding to the 3′-untranslated region (UTR) of a target gene and is processed from introns, exons, or intergenic regions. A mature miRNA duplex is incorporated as a single-stranded RNA with the Argonaute protein into an RNA-induced silencing complex (RISC), which leads to either cleavage or translational inhibition of a target mRNA.

In addition, extracellular vehicles (EVs) are vesicles that cells export out of the cell, have a lipid bilayer structure, and are substances responsible for interactions between cells. Further, EVs contain metabolites such as proteins and nucleic acids from cells from which they originate, and play an important role in signaling between cells. Recently, they have been attracting attention as a material and drug delivery vehicle for treatment and regeneration through a process of inserting specific substances into EVs.

However, in relation to the prevention or treatment of colon cancer, the specific therapeutic effect of extracellular vesicles containing miRNA on adipose-derived stem cell-derived colon cancer-related target substances has not yet been reported.

### RELATED ART DOCUMENT

### Patent Document Korean Patent Application Publication No. 10-2015-0100335

### SUMMARY OF THE INVENTION

### Technical Problem

It is an object of the present disclosure to provide a pharmaceutical composition for preventing or treating colon cancer comprising extracellular vesicles loaded with an MACC1 inhibitor as an active ingredient.

It is another object of the present disclosure to provide a kit for preventing or treating colon cancer comprising: a composition comprising extracellular vesicles loaded with an MACC1 inhibitor as an active ingredient; and an instruction therefor.

However, the technical problem to be achieved by the present disclosure is not limited to the problems mentioned above, and other problems not mentioned can be clearly understood by those skilled in the art from the following description.

### Technical Solution

The present disclosure provides a pharmaceutical composition for preventing or treating colon cancer comprising extracellular vesicles loaded with an MACC1 inhibitor as an active ingredient.

In an embodiment of the present disclosure, the extracellular vesicles may comprise a TM4SF5-targeting peptide, but are not limited thereto.

In an embodiment of the present disclosure, the peptide may comprise SEQ ID NO: 1, but is not limited thereto.

In an embodiment of the present disclosure, the inhibitor may be one selected from the group consisting of miRNA, short hairpin RNA (shRNA), small interference RNA (siRNA), ribozyme, DNAzyme, PNA, an antibody, and an aptamer, but is not limited thereto.

In an embodiment of the present disclosure, the inhibitor may be miR143, but is not limited thereto.

In an embodiment of the present disclosure, the miR143 may comprise SEQ ID NO: 3, but is not limited thereto.

In an embodiment of the present disclosure, the extracellular vesicles may be derived from adipose-derived stem cells, but are not limited thereto.

In an embodiment of the present disclosure, the composition may be administered intravenously, but is not limited thereto.

In accordance with another aspect of the present disclosure, there is provided a kit for preventing or treating colon cancer, the kit comprising: a composition comprising extracellular vesicles loaded with an MACC1 inhibitor as an active ingredient and an instruction therefor.

In an embodiment of the present disclosure, the extracellular vesicles may comprise a TM4SF5-targeting peptide, but are not limited thereto.

In accordance with still another aspect of the present disclosure, there is provided use of extracellular vesicles loaded with an MACC1 inhibitor or a composition comprising the extracellular vesicles as an active ingredient for the prevention or treatment of colon cancer.

In accordance with still another aspect of the present disclosure, there is provided use of extracellular vesicles loaded with an MACC1 inhibitor or a composition comprising the extracellular vesicles as an active ingredient for preparing a preparation for the prevention or treatment of colon cancer.

In accordance with yet another aspect of the present disclosure, there is provided a method of preventing or treating colon cancer, the method comprising: administering extracellular vesicles loaded with an MACC1 inhibitor or a composition comprising the extracellular vesicles as an active ingredient to a subject in need thereof.

### Advantageous Effects

The pharmaceutical composition for preventing or treating colon cancer including extracellular vesicles loaded with an MACC1 inhibitor as an active ingredient allows the extracellular vesicles loaded with the MACC1 inhibitor, a colon cancer-promoting factor, to deliver the inhibitor specifically to colon cancer cells and is characterized by high stability and biodegradability, thereby having a low risk of side effects and excellent drug delivery. Therefore, the pharmaceutical composition of the present disclosure is expected to be utilized in a variety of ways as a treatment for colon cancer with minimal risk of side effects and maximized anticancer ability.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1A is a schematic diagram illustrating a method of manufacturing TM4SF5-targeting extracellular vesicles loaded with MACC1 miRNA of the present disclosure;

FIG. 1B is a graph illustrating the results of measuring the size of TM4SF5-targeting extracellular vesicles loaded with MACC1 miRNA of the present disclosure;

FIG. 1C is a photograph illustrating a TEM imaging result of extracellular vesicles of the present disclosure;

FIG. 2 is a set of graphs illustrating MACC1 and apoptosis inhibition effects confirmed by real-time PCR after treating a colon cancer cell line with the TM4SF5-targeting extracellular vesicles loaded with MACC1 miRNA of the present disclosure;

FIG. 3 is a set of photos illustrating the MACC1 and apoptosis inhibition effects confirmed by Western blot after treating colon cancer cells line with the extracellular vesicles of the present disclosure alone or with the TM4SF5-targeting extracellular vesicles loaded with MACC1 miRNA of the present disclosure;

FIG. 4 is a set of photographs and graphs illustrating cell migration ability taken after treating colon cancer cells line with the extracellular vesicles of the present disclosure alone or with the TM4SF5-targeting extracellular vesicles loaded with MACC1 miRNA of the present disclosure;

FIG. 5 is a set of images and graphs illustrating the degree of tumor targeting of the extracellular vesicles confirmed by IVIS in-vivo imaging after treating colon cancer mouse models with the extracellular vesicles of the present disclosure alone or with the TM4SF5-targeting extracellular vesicles loaded with MACC1 miRNA of the present disclosure;

FIGS. 6A and 6B are a set of photographs and graphs illustrating changes in tumor size confirmed after treating colon cancer mouse models with the extracellular vesicles of the present disclosure alone or with the TM4SF5-targeting extracellular vesicles loaded with MACC1 miRNA of the present disclosure;

FIG. 7 is a set of graphs illustrating changes in apoptosis, confirmed by real-time PCR, within the tumor tissues obtained after treating colon cancer mouse models with the extracellular vesicles of the present disclosure alone or with the TM4SF5-targeting extracellular vesicles loaded with MACC1 miRNA of the present disclosure;

FIG. 8 is a set of images illustrating changes in apoptosis, confirmed by western blot, within the tumor tissues obtained after treating colon cancer mouse models with the extracellular vesicles of the present disclosure alone or with the TM4SF5-targeting extracellular vesicles loaded with MACC1 miRNA of the present disclosure; and

FIGS. 9A and 9B are a set of images and graphs illustrating changes in apoptosis, confirmed by immunostaining (IHC), within the tumor tissues obtained after treating colon cancer mouse models with the extracellular vesicles of the present disclosure alone or with the TM4SF5-targeting extracellular vesicles loaded with MACC1 miRNA of the present disclosure. Specifically, the images and graphs show that the expression of BCL-xL and Bax decrease and increase, respectively.

### DETAILED DESCRIPTION OF THE INVENTION

The present disclosure provides a pharmaceutical composition for preventing or treating colon cancer comprising extracellular vesicles loaded with an MACC1 inhibitor as an active ingredient.

In the present disclosure, “metastasis-associated colon cancer 1 (MACC1)” was first identified in colon cancer by differential display RT-PCR and may be involved in regulating colon tumor growth and metastasis through hepatocyte growth factor (HGF)/c-Met, without being limited thereto.

In the present disclosure, “inhibitor” may include anything that inhibits the expression of a specific substance, especially a protein, or suppresses the activity of its function. Accordingly, the MACC1 inhibitor of the present disclosure may include all substances that inhibit the expression of MACC1 protein or inhibit MACC1 protein activity, or substances that inhibit MACC1 protein expression and activity.

The present disclosure was completed by confirming that extracellular vesicles loaded with the inhibitor of MACC1, a colon cancer-promoting factor, can exert excellent colon cancer treatment effects by delivering the inhibitor specifically to colon cancer cells. The extracellular vesicles of the present disclosure are a drug carrier with a low risk of side effects due to high stability and biodegradability, and excellent drug delivery ability. It was confirmed that lipid nanoparticles loaded with MACC1-specific miRNA can effectively deliver the miRNA to colon cancer cells and induce inhibition of MACC1 expression, and even when administered to a colon cancer animal model, they travel to tumors to deliver MACC1 expression-inhibiting miRNA thereto. In particular, it was confirmed that when an MACC1 inhibitor is combined with a colon cancer cell-targeting peptide and mounted on the extracellular vesicles, the delivery of the MACC1 inhibitor to colon cancer cells is further increased, further enhancing the anticancer effect through MACC1 inhibition. Accordingly, the extracellular vesicles loaded with the MACC1 inhibitor according to the present disclosure are expected to be used in a variety of ways as a colon cancer treatment with minimal risk of side effects and maximized anticancer ability.

In an embodiment of the present disclosure, the extracellular vesicles may comprise a transmembrane 4 L6 family member 5 (TM4SF5)-targeting peptide, but is not limited thereto.

In addition, the present disclosure, the TM4SF5-targeting peptide may be bound to the surface of the extracellular vesicles, but is not limited thereto.

In the present disclosure, “transmembrane 4 L6 family member 5 (TM4SF5)” is a protein encoded by the TM4SF5 gene and is a member of the transmembrane 4 superfamily also known as the tetraspanin family. Most of the members of the superfamily may be cell surface proteins characterized by the presence of four hydrophobic domains. In addition, TM4SF5 can mediate signaling processes that play a role in the regulation of cell development, activation, growth and motility, but is not limited thereto.

In an embodiment of the present disclosure, the peptide may comprise SEQ ID NO: 1, but is not limited thereto.

The peptide may comprise SEQ ID NO: 1 and, for example, may be represented by SEQ ID NO: 1. SEQ ID NO: 1 may be encoded by the nucleotide sequence of SEQ ID NO: 2, but is not limited thereto.

In an embodiment of the present disclosure, the inhibitor may be one selected from the group consisting of miRNA, short hairpin RNA (shRNA), small interference RNA (siRNA), ribozyme, DNAzyme, PNA, an antibody, and an aptamer, but is not limited thereto.

In an embodiment of the present disclosure, the inhibitor may be miR143, but is not limited thereto.

In an embodiment of the present disclosure, the miR143 may comprise SEQ ID NO: 3, but is not limited thereto.

The miR143 may comprise SEQ ID NO: 3 and, for example, may be represented by SEQ ID NO: 3, but is not limited thereto.

miRNA is a small non-coding RNA consisting of 18-25 nucleotides (nt) that regulates gene expression by binding to the 3′-untranslated region (UTR) of a target gene and is processed from introns, exons or intergenic regions. First, miRNA is transcribed into pri-miRNA molecules containing thousands of nucleotides by RNA polymerase, and the pri-miRNA is subsequently processed by a microprocessor to form an approximately 70 nt stem-loop intermediate known as a miRNA precursor. Next, pre-miRNA is transported from the nucleus to the cytoplasm via Exportin-5 (EXP5) and cofactor Ran-GTP. Here, the pre-miRNA is processed into an 18-25 nt mature miRNA duplex by the RNase endonuclease Dicer. A mature miRNA duplex is incorporated as a single-stranded RNA with the Argonaute protein into the RNA-induced silencing complex (RISC), which induces either cleavage or translational inhibition of a target mRNA.

In the present disclosure, the sequence of the miRNA may be the seed sequence of the corresponding miRNA. “Seed sequence” refers to the nucleotide sequence of a certain region within a miRNA that binds with complete complementarity when miRNA recognizes a target. This is an essential part for miRNA to bind to a target and is a site that performs an actually effective function. In other words, miRNAs used in the present disclosure are a concept including functional equivalents of the nucleic acid molecules constituting them, e.g., variants capable of performing a functionally equivalent effect to the miRNA nucleic acid molecule even if some base sequences of the miRNA nucleic acid molecule are modified by deletion, substitution or insertion.

In the present disclosure, the terms “percent identity,” “sequence identity,” “percent similarity,” “sequence similarity,” “percent sequence identity,” and the like, as used herein in relation to amino acid sequences and/or nucleic acid sequences, may refer to a measure determined by maximizing the similarity between aligned amino acid residues or nucleotides and comparing the degree of similarity of two sequences based on an alignment of the sequences, which is a function of the number of identical or similar residues or nucleotides, the total number of residues or nucleotides, and the presence and length of gaps in the sequence alignment, but is not limited thereto.

A portion of a polynucleotide or polypeptide sequence may contain an addition or a deletion (i.e., a gap) relative to a reference sequence (which does not contain an addition or a deletion), for optimal alignment of two sequences. The percentage is calculated by determining the number of positions where the same nucleic acid base or amino acid residue appears in both sequences, dividing the number of matching positions by the total number of positions in the comparison window, and multiplying the result by 100 to yield the percent sequence identity.

The term “identical” or percentage “identity” with respect to two or more nucleic acids or polypeptide sequences means two or more sequences or sub-sequences that are identical or have a specified percentage (i.e., approximately 60% identity, preferably 65%, 70%, 75%, 80%, 85%, 90%%, 91%, 92%, 93%, 94%, 95%, 96%, 97%, 98%, 99% or higher identity, for the specified regions when compared and sorted for maximum correspondence to the comparison window or displayed regions) of identical amino acid residues or nucleotides as determined using the BLAST or BLAST 2.0 sequence comparison algorithm using the default parameters described below or by manual alignment and visual inspection (for example, see NCBI website http://www.ncbi.nlm.nih.gov/BLAST/etc.). These sequences are then referred to as “substantially identical.”.

This definition may also refer to or be applied to the complementation of a test sequence. The definition also includes sequences with deletions and/or additions as well as those with substitutions. As described below, the preferred algorithm can account for gaps, etc. Preferably, the identity exists over a region that is at least about 25 amino acids or nucleotides in length, or more preferably over a region that is 50 to 100 amino acids or nucleotides in length.

Amino acid or nucleotide base “positions” are indicated by numbers that sequentially identify each amino acid (or nucleotide base) in the reference sequence based on its position relative to the N-terminus (or 5′-end). Because of deletions, insertions, truncations, fusions, etc., which must be considered when determining the optimal alignment, the number of amino acid residues in a test sequence determined by simply counting from the N-terminus will not necessarily be the same as the number of its corresponding position in the reference sequence. For example, if a variant has a deletion relative to an aligned reference sequence, there will be no amino acid in the variant that corresponds to the position in the reference sequence at the site of the deletion. If there is an insertion in the aligned reference sequence, the insertion will not correspond to a numbered amino acid position in the reference sequence. In the case of cleavage or fusion, there may be an extension of an amino acid in either the reference or alignment sequence that does not correspond to any amino acid in the corresponding sequence.

When used in connection with the numbering of a given amino acid or polynucleotide sequence, the term “numbered with respect to” or “corresponding to” refers to the numbering of residues in a reference sequence specified when a given amino acid or polynucleotide sequence is compared to the reference sequence.

In the present disclosure, the miRNA may be a mature miRNA as described, but may be miRNA precursor, pri-miRNA or plasmid-type miRNA precursor.

In the present disclosure, the miRNA may exist in single-stranded or double-stranded form. Mature miRNA molecules exist primarily as single strands, but precursor miRNA molecules may contain partially self-complementary structures (e.g., stem-loop structures) that can form double-strands.

In addition, at least one of the nucleic acid molecules constituting the miRNA of the present disclosure may have a form containing a phosphorothiolate structure in which the phosphate backbone structure is substituted with a sulfur element; a form substituted with DNA, peptide nucleic acids (PNA) or locked nucleic acid (LNA) molecules; or a form where the 2′ hydroxyl group of a sugar is substituted with a methylated, methoxylated, or fluorinated structure.

The miRNAs of the present disclosure may be isolated or prepared using standard molecular biology techniques, such as chemical synthesis methods or recombinant methods, or commercially available miRNAs may be used.

In the present disclosure, the miRNA nucleic acid molecule may be contained in an expression vector. The expression vector is preferably one that encodes the miRNA of the present disclosure in an expressible form and can be introduced into a host according to a method commonly used in the art. Here, in the present disclosure, the expression vector may be used without limitation as long as it is intended to deliver miRNA. In addition, In the present disclosure, the expression vector may preferably additionally contain a selection marker to facilitate the selection of transformed cells, but is not limited thereto. In the present disclosure, the expression vector may be introduced into a host cell to provide a transformed transformant, but is not limited thereto.

In an embodiment of the present disclosure, the extracellular vesicles may be derived from adipose-derived stem cells, but are not limited thereto.

In the present disclosure, to isolate miRNA from adipose-derived stem cells, stem cells may be pretreated, stored, and cultured using methods commonly performed in the art. For example, to extract miRNA from adipose-derived stem cells with high yield, the cells can be pretreated and stored using a specific method, and to facilitate culturing or to contain the miRNA at a high level of concentration, functional substances may be added, without being limited thereto.

In an embodiment of the present disclosure, the composition may be administered intravenously, but is not limited thereto.

In the present disclosure, the inhibitor may be combined with a cell-penetrating peptide, but is not limited thereto.

In the present disclosure, the extracellular vesicles may have a diameter of 50 to 500 nm, but are not limited thereto.

In the present disclosure, the colon cancer may include all cancers included in colon cancer regardless of a specific part of the large intestine, and, for example, may be right colon cancer, left colon cancer, rectal cancer, etc., without being limited thereto.

The “pharmaceutical composition” according to the present disclosure may further include suitable carriers, excipients, and diluents which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled-release additive.

The pharmaceutical composition according to the present disclosure may be formulated into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols according to a commonly used respective method. The preparation for external use may have a formulation such as creams, gels, patches, sprays, ointments, plasters, lotions, liniments, pastes, or cataplasmas.

Examples of a carrier, excipient, and diluent that may be contained in the pharmaceutical composition according to the present disclosure includes lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

When formulated, a commonly used diluent or excipient, such as a filler, an expander, a binder, a wetting agent, a disintegrant, a surfactant, and the like, may be used for preparation.

As additives of tablets, powders, granules, capsules, pills, and troches according to the present disclosure, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ionexchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present disclosure, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present disclosure, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present disclosure, purified water may be used, and as necessary, an emulsifier, a preservative, a stabilizer, a fragrance, or the like may be used.

In suspensions according to the present disclosure, suspending agents such as acacia, tragacanth, methylcellulose, carboxymethylcellulose, sodium carboxymethylcellulose, microcrystalline cellulose, sodium alginate, hydroxypropyl methylcellulose, HPMC 1828, HPMC 2906, HPMC 2910, and the like may be used, and as necessary, a surfactant, a preservative, a stabilizer, a colorant, and a fragrance may be used . . .

Injections according to the present disclosure may include solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO3) carbon dioxide gas, sodium metabisulfite (Na2S2O5), sodium sulfite (Na2SO3), nitrogen gas (N2), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present disclosure, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75 (S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and these solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Nonlimiting examples of the non-aqueous solvent and the suspension include propylene glycol, polyethylene glycol, a vegetable oil such as olive oil, and an injectable ester such as ethyl oleate.

The pharmaceutical composition according to the present disclosure is administered in a pharmaceutically effective amount. In the present disclosure, “pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well-known in other medical fields.

The pharmaceutical composition according to the present disclosure may be administered as an individual therapeutic agent, may be administered in combination with other therapeutic agents, may be administered sequentially or simultaneously with conventional therapeutic agents, and may be administered once or multiple times. In consideration of all of the above factors, it is important to administer an amount capable of obtaining the maximum effect with a minimum amount without side effects, which can easily be determined by those skilled in the art to which the present disclosure pertains.

The pharmaceutical composition of the present disclosure may be administered to a subject by various routes. All modes of administration may be considered, for example, oral administration, subcutaneous injection, intraperitoneal administration, intravenous injection, intramuscular injection, paraspinal space (intrathecal) injection, sublingual administration, buccal administration, rectal insertion, vaginal insertion, ocular administration, otic administration, nasal administration, inhalation, spray through the mouth or nose, dermal administration, transdermal administration, etc.

The pharmaceutical composition of the present disclosure is determined according to the type of drug as an active ingredient along with several related factors such as the disease to be treated, the route of administration, the patient's age, sex, weight, and severity of the disease. Specifically, the effective amount of the composition according to the present disclosure may vary depending on the patient's age, gender, and weight, and is generally administered at 0.001 to 150 mg, preferably 0.01 to 100 mg, per kg of body weight daily or every other day, or divided into 1 to 3 times a day. However, since the dosage may increase or decrease depending on the route of administration, severity of disease, gender, weight, age, etc., the dosage does not limit the scope of the present disclosure in any way.

In the present disclosure, As used herein, the term “subject” refers to a subject in need of treatment for a disease, and more specifically, may be a mammal of humans or non-human primates, mice, rats, dogs, cats, horses, cattle, etc., but is not limited thereto.

In the present disclosure, “administration” refers to provision of a predetermined composition of the present disclosure to a subject by any suitable method. In the present disclosure, “prevention” refers to all actions that inhibit or delay the onset of a target disease; “treatment” refers to all actions that improve or beneficially change a target disease and metabolic abnormalities thereof by the administration of the pharmaceutical composition according to the present disclosure; and “alleviation” refers to all actions that reduce target disease-related parameters, for example, the degree of symptoms, by the administration of the pharmaceutical composition according to the present disclosure.

The present disclosure provides a kit for preventing or treating colon cancer including a composition comprising extracellular vesicles loaded with an MACC1 inhibitor as an active ingredient and an instruction therefor.

In an embodiment of the present disclosure, the extracellular vesicles may comprise a TM4SF5-targeting peptide, but are not limited thereto.

The “kit” of the present disclosure may include, in addition to the composition, other components, devices, materials, etc. commonly required for methods to enhance the colon cancer treatment effect of the extracellular vesicles of the present disclosure or to store or manage the extracellular vesicles of the present disclosure. In addition, all components included in the kit may be used one or more times without limitation, there are no restrictions on the order or timing of using each substance, and the application of each substance may be carried out simultaneously or in small steps.

The kit of the present disclosure may include a container in addition to the formulation and an instruction therefor. The container may serve to package the components and may also serve to store and secure them. The material of the container may take the form of, for example, a bottle, tub, sachet, envelope, tube, ampoule, etc., which may be partially or entirely made of plastic, glass, paper, foil, wax, etc. The container may be equipped with a completely or partially removable closure that may initially be a part of the container or may be attached to the container by mechanical, adhesive, or other means, and may be equipped with a stopper that allows access to contents therein by means of a syringe needle. The kit may contain an external package, and the external package may contain instructions for use of the components, without being limited thereto.

In addition, the present disclosure provides a method of preparing extracellular vesicles loaded with an MACC1 inhibitor, the method comprising the following steps:
a step of cloning adipose-derived stem cells into a vector containing a gene encoding a TM4SF5-targeting peptide;
a step of extracting extracellular vesicles from the adipose-derived stem cells; and
a step of introducing an MACC1 inhibitor into the extracellular vesicles.

Now, the present disclosure will be described in more detail with reference to the following preferred examples. These examples are provided for illustrative purposes only and should not be construed as limiting the scope and spirit of the present disclosure.

### EXAMPLES

### Example 1. Manufacture and Confirmation of Extracellular Vesicles Loaded with MACC1 miRNA

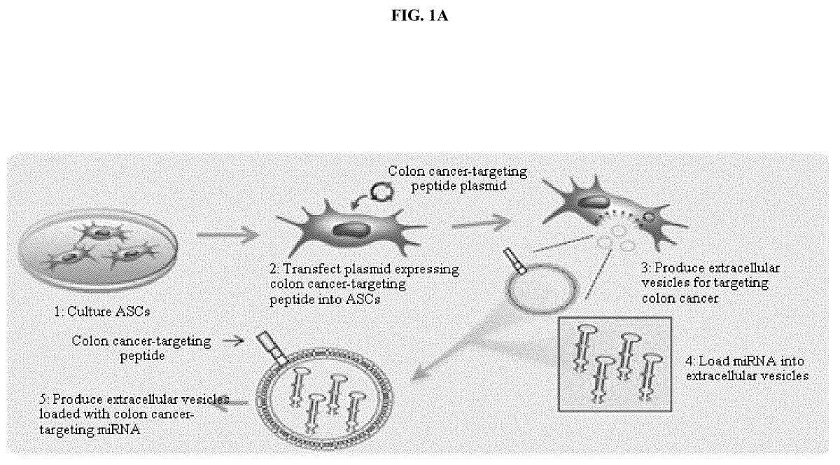

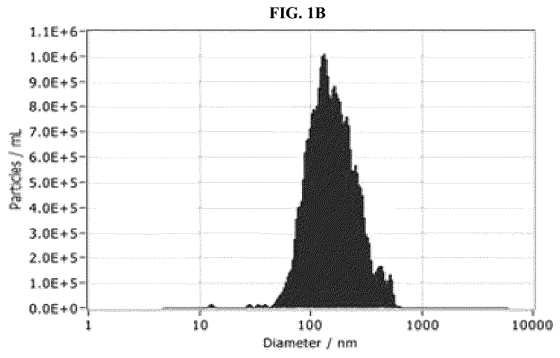

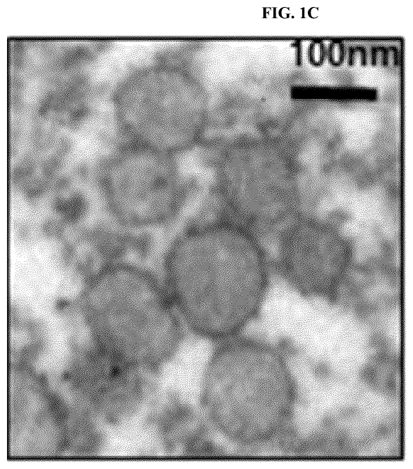

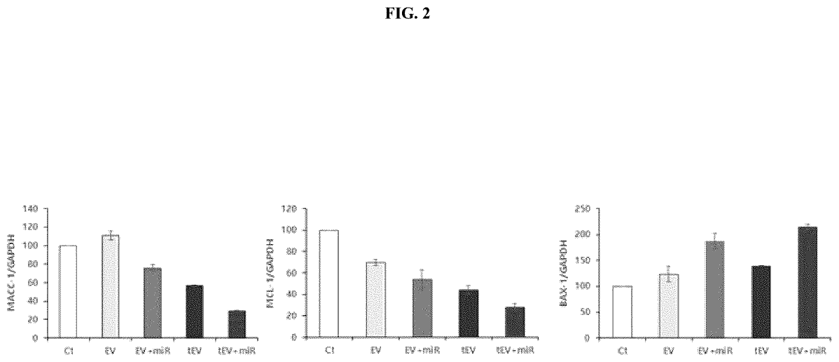

Extracellular vesicles loaded with miRNA that can suppress the expression of MACC1 were manufactured (FIG. 1A). Since various conventional nanoparticle delivery vehicles can cause toxicity or side effects when administered into the human body, a delivery vehicle that ensures stability was created by producing nanoparticles in cells. Specifically, TM4SF5 targeting peptide in pDisplay vector was transduced into adipose-derived stem cells using a cloning method, and then extracellular vesicles were extracted using tangential flow filtering (TFF). Next, miR143 was introduced into extracellular vesicles extracted using the Exo-fect exosome transfection kit.

To check the size and number of particles of the extracellular vesicles, they were observed using Zetaview. As a result, it was confirmed as shown in FIG. 1B that the size of the extracellular vesicles according to the present disclosure was constant and the peak appeared uniformly. In addition, the shape of extracellular vesicles was observed through TEM imaging. As a result, it was confirmed as shown in FIG. 1C that the shape of the extracellular vesicles according to the present disclosure appeared consistently.

### Example 2. Confirmation of MACC1 Expression and Apoptosis Inhibition Effect of Extracellular Vesicles Loaded with MACC1 miRNA Through PCR Analysis (In Vitro): Validation Experiment

Whether the extracellular vesicles loaded with the MACC1-inhibiting miRNA manufactured in Example 1 deliver the miRNA to cells and inhibit the expression of the target gene MACC1 and apoptosis-related genes were analyzed. To confirm this, the expression levels of MACC1 were confirmed by real-time PCR after treating each of colon cancer cell lines with extracellular vesicles not loaded with miRNA or extracellular vesicles loaded with MACC1-inhibiting miRNA. Colon cancer cell lines such as HCT116 and HT29 were used.

TABLE 1

| | EV | Target EV | miR EV | miR target EV |
|---|---|---|---|---|
| Classification | group | group | group | group |
| Treatment condition | 5 × 10⁶ | 5 × 10⁶ | 5 × 10⁶ | 5 × 10⁶ |
| (particles) | | | | |

As a result, it was confirmed as shown in FIG. 2 that the groups treated with extracellular vesicles loaded with MACC1-inhibiting miRNA showed a significant decrease in MACC1 expression compared to the untreated control group. These results suggest that the extracellular vesicles of the present disclosure can effectively deliver the MACC1-inhibiting miRNA to colon cancer cells and effectively inhibit the expression of the oncogene MACC1.

In addition, it was confirmed that the expression level of Bax, a pro-apoptotic factor, increased, and the expression of MCL-1, an anti-apoptotic factor, decreased most in the group treated with extracellular vesicles loaded with MACC1-inhibiting miRNA.

### Example 3. Confirmation of MACC1 Expression and Apoptosis Inhibition Effect of Extracellular Vesicles Loaded with MACC1 miRNA Through Western Blot Experiment (In Vitro)

The effect of MACC1 expression and apoptosis inhibition in Example 2 was analyzed through a western blot experiment. Here, the specific experimental method was carried out in the same manner as in Example 2.

Protein was extracted from HCT116 colon cancer cells using the RIPA Lysis kit. The extracted protein was quantified using the Bradford assay and 20 ug of each protein was used. The extracted protein was reacted with a primary antibody (1:1,000 dilution) at 4° C. for 24 hours, and then reacted with horseradish peroxidase (HRP)-conjugated secondary antibody (1:2,000 dilution) at room temperature for 1 hour, and then detected using Western Blotting Plus Chemiluminescence Reagent (Millipore, Bedford, MA, USA).

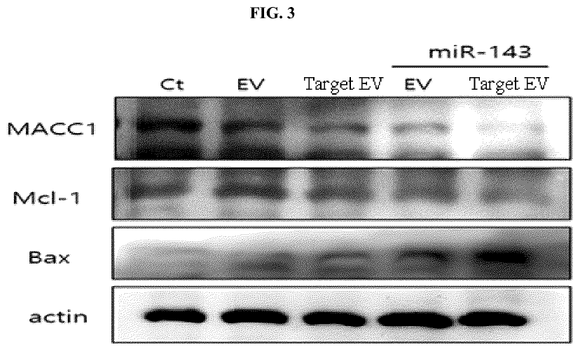

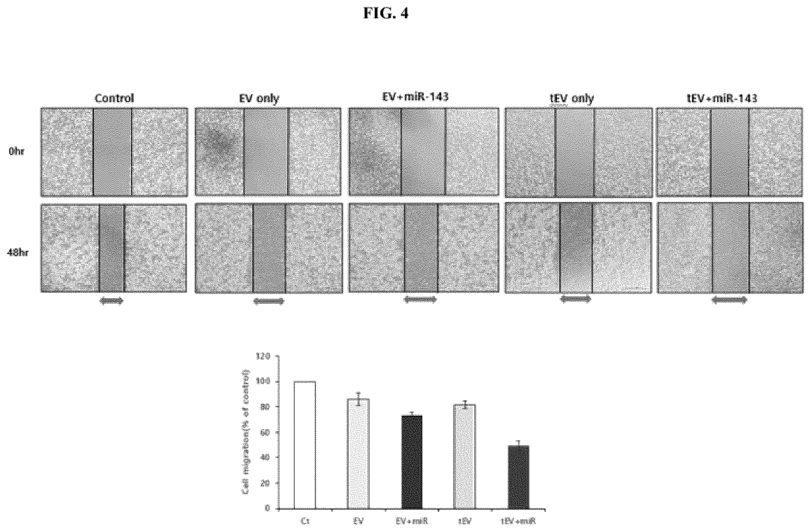

As a result, the Western blot experiment results were confirmed to correspond to Example 2. Specifically, in the group treated with extracellular vesicles loaded with MACC1-inhibiting miRNA, MACC1 expression was significantly reduced and Bax was increased, while MCL-1 expression was most decreased, as shown in FIG. 3.

### Example 4. Confirmation of Inhibitory Effect of Extracellular Vesicles Loaded with MACC1 miRNA on Intercellular Movement

The intercellular migration ability of the extracellular vesicles loaded with the MACC1 inhibitor prepared in Example 1 was analyzed. Specifically, HCT116 cells were cultured in 48 wells and scratched, and non-targeted extracellular vesicles and target endoplasmic reticulum with suppressed MACC1 expression were photographed before and 48 hours after treatment, respectively, to confirm cell migration.

As a result, it was confirmed that the extracellular vesicles encapsulated with the MACC1 expression-inhibiting miRNA targeting colon cancer according to the present disclosure increased the effect of inhibiting migration, compared to extracellular vesicles loaded only with MACC1 expression-inhibiting miRNA (FIG. 4).

### Example 5. Confirmation of Targetability of Extracellular Vesicles Loaded with MACC1 miRNA in Mouse Model

To analyze the targetability of the extracellular vesicles prepared in Example 1, it was investigated whether the extracellular vesicles encapsulated with the MACC1-inhibiting miRNA loaded with the colon cancer-targeting peptide could migrate to tumor tissue when administered to mice. Specifically, colon cancer mouse models were created by directly injecting a colon cancer cell line into the buttocks of 5-6-week-old mice (BALB/c nude mice). 2×10⁶ particles of the extracellular vesicles produced in Example 1 were administered to the mice by intravenous injection of 2×106 particles twice a week for 3 weeks, and their movement was observed using IVIS in vivo imaging equipment.

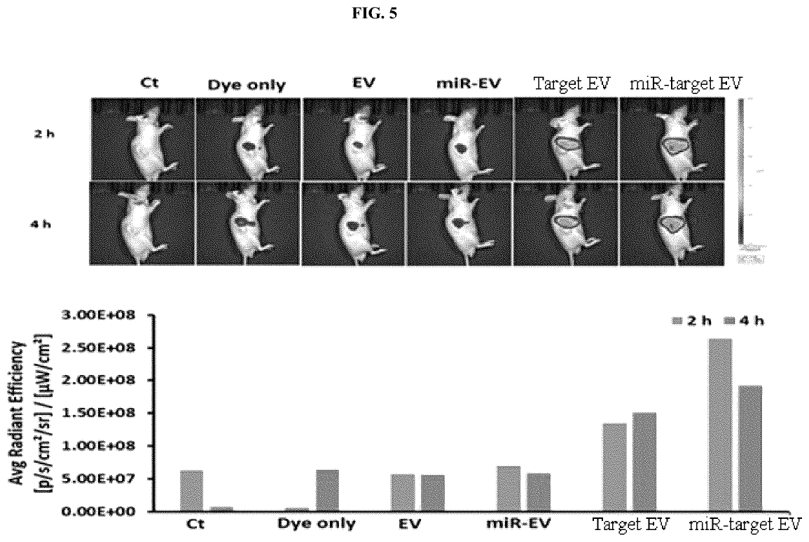

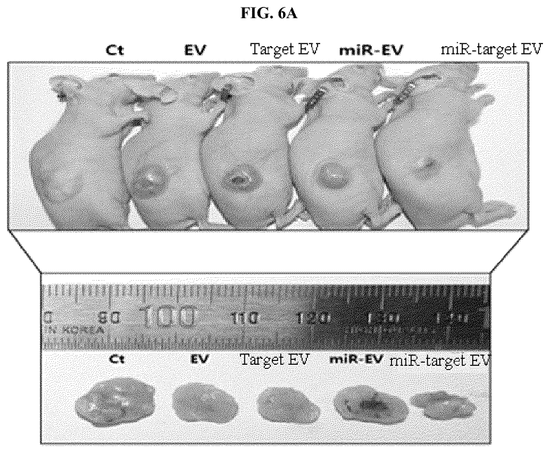

As a result, it was confirmed as shown in FIG. 5 that migration to tumors was found to increase in the group administered with the colon cancer-targeting MACC1-inhibiting miRNA, and the colon cancer-targeting extracellular vesicles of the present disclosure could be effectively delivered to tumor cells when administered into the body.

### Example 6. Confirmation of Anti-Tumor Activity of Extracellular Vesicles Loaded with MACC1 miRNA in Mouse Model

To analyze the targetability of the extracellular vesicles manufactured in Example 1, it was confirmed whether extracellular vesicles encapsulated with MACC1-inhibiting miRNA loaded with a colon cancer-targeting peptide could migrate to tumor tissue when administered to mice. The specific mouse model, experimental conditions, and experimental method were the same as in Example 5. Next, the sizes of the tumors in the colon cancer mouse models were measured.

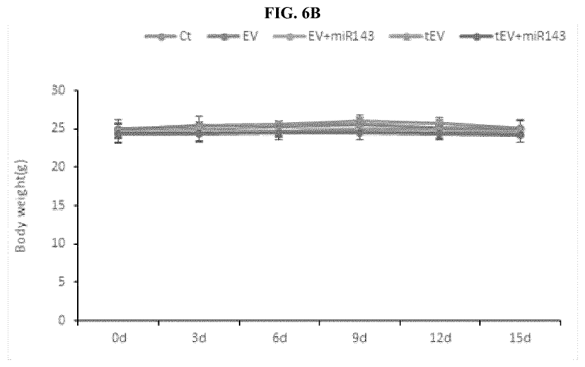

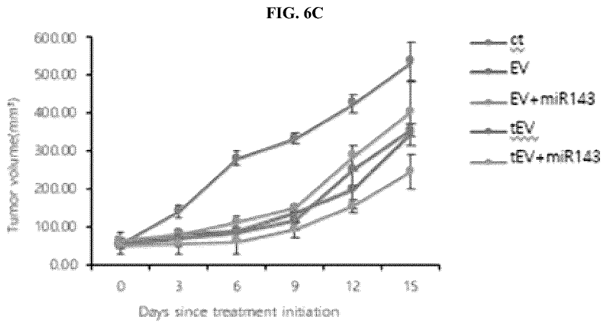

As a result of investigating the sizes of the tumors in the colon cancer mouse models, it was confirmed that the tumor was most suppressed in the group injected with the extracellular vesicles encapsulated with the MACC1-inhibiting miRNA loaded with the colon cancer-targeting peptide (FIG. 6).

### Example 7. Confirmation of MACC1 Expression and Apoptosis Inhibition Effect of Extracellular Vesicles Loaded with MACC1 miRNA Through PCR Analysis (In Vivo)

To investigate the antitumor activity of the extracellular vesicles manufactured in Example 1, whether the MACC1 expression and apoptosis inhibition effects shown in Examples 2 and 3 were also observed in vivo was analyzed. The specific mouse model was prepared in the same way as Example 5, and the experimental conditions and experimental methods for the expression of MACC1 and apoptosis-related factors were conducted in the same way as in Example 2.

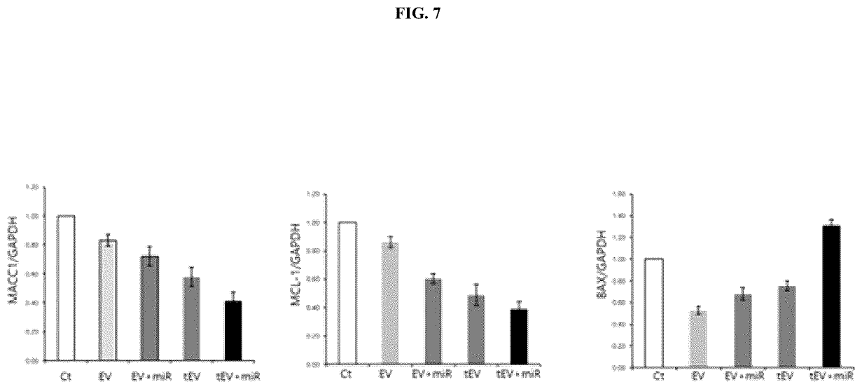

As a result, in the group injected with extracellular vesicles encapsulated with the MACC1-inhibiting miRNA loaded with the colon cancer-targeting peptide, it was confirmed through real-time PCR that the inhibition of MACC1 expression and the expression of Bax, a pro-apoptotic factor, tended to increase the most, and the expression of MCL-1, an anti-apoptotic factor, tended to decrease the most (FIG. 7).

### Example 8. Confirmation of MACC1 Expression and Apoptosis Inhibition Effect of Extracellular Vesicles Loaded with MACC1 miRNA Through Western Blot Experiment (In Vivo)

The in-vivo antitumor activity of the extracellular vesicles of the present disclosure confirmed in Example 7 was reconfirmed through Western blot experiments. The specific mouse model was prepared in the same way as Example 5, and the experimental conditions and experimental methods for the expression of MACC1 and apoptosis-related factors were conducted in the same way as in Example 3.

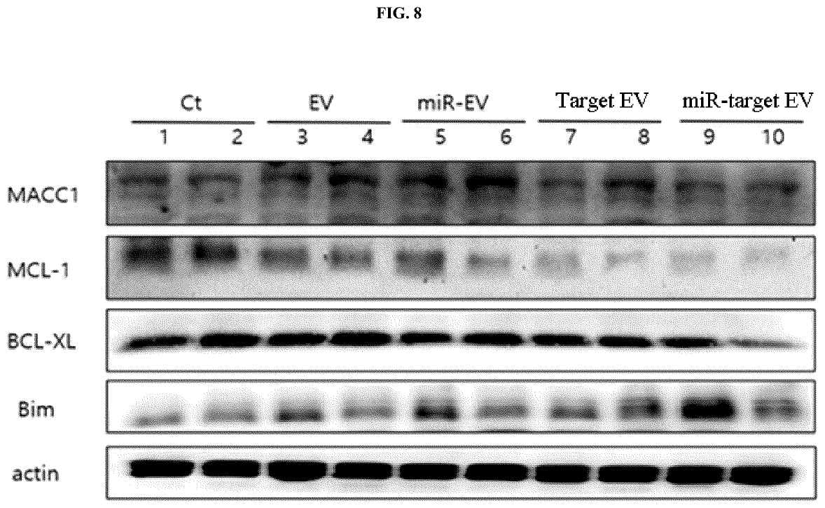

As a result, in the group injected with extracellular vesicles encapsulated with the MACC1-inhibiting miRNA loaded with the colon cancer-targeting peptide, it was confirmed through Western blot experiments that the inhibition of MACC1 expression and the expression of BIM, a pro-apoptotic factor, tended to increase the most, and the expression of MCL-1 and BCL-XL, anti-apoptotic factors, tended to decrease the most (FIG. 8).

### Example 9. Confirmation of Apoptosis Inhibition Effect of Extracellular Vesicles Loaded with MACC1 miRNA Through IHC Analysis (In Vivo)

The in-vivo antitumor activity of the extracellular vesicles of the present disclosure confirmed in Examples 6 and 7 was reconfirmed through Western blot experiments. A specific mouse model was prepared in the same manner as in Example 5. In addition, for IHC analysis, mouse paraffin tissue was sectioned and attached to a slide, and the paraffin was melted in a slide warmer. Next, paraffin was removed from xylene, and xylene was removed in 100-70% EtOH. Next, after performing antigen retrieval in 1x Citrate Buffer (pH6.0), primary and secondary antibodies were sequentially combined and a substate was added thereto for color development. Next, the nuclei were stained, mounted and scanned, and the results were confirmed.

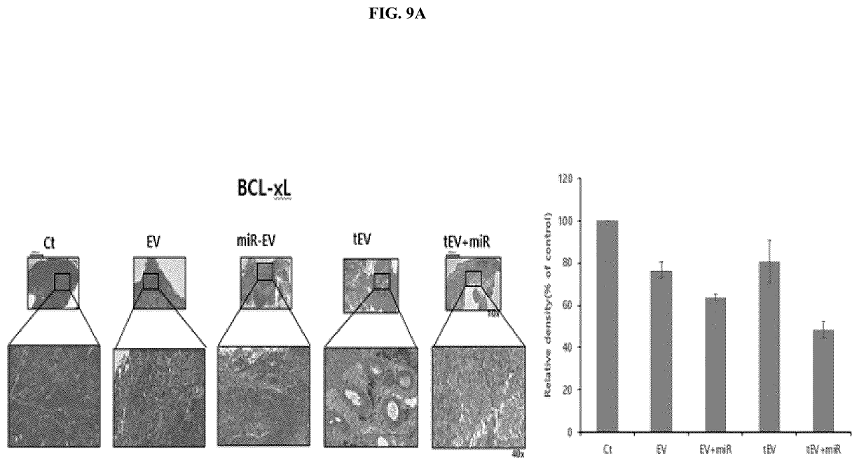

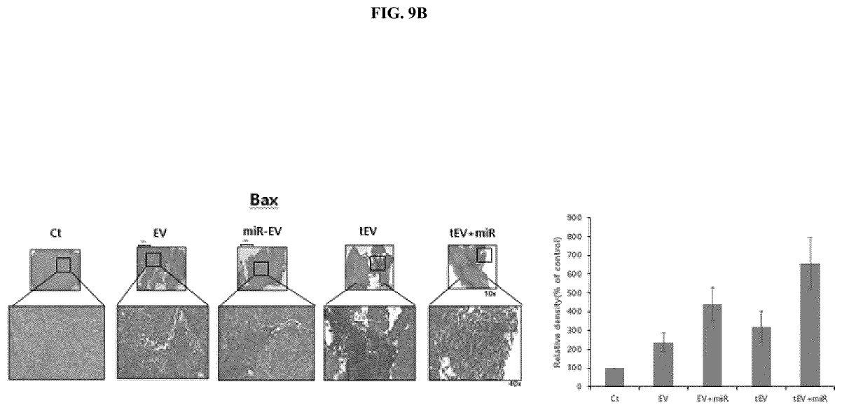

As a result, in the group injected with the extracellular vesicles encapsulated with the MACC1-inhibiting miRNA loaded with the colon cancer-targeting peptide, it was confirmed through IHC analysis that the expression of BCL-XL, an anti-apoptotic factor, decreased (FIG. 9A), and the expression of Bax, a pro-apoptotic factor, tended to increase the most (FIG. 9B).

TABLE 2

| Sequence | | |
|---|---|---|
| number | Name | Sequence |
| 1 | TM4SF5-specific | TAGAYLLNRTLWDRCEAPPRVVPWNVT |
| 2 | peptide | ACA GCA GGA GCA TAC TTG TTG AAT AGA |
| | | ACC CTG TGG GAC CGA TGC GAA GCT CCA |
| | | CCC CGA GTA GTT CCG TGG AAT GTT ACT |
| 3 | miR-143 miRNA | GGU GCA GUG CUG CAU CUC UGG U |
| | (BIONEER, NO. | |
| | O-211001-0046-R) | |

The aforementioned description of the present disclosure is provided by way of example and those skilled in the art will understand that the present disclosure can be easily changed or modified into other specified forms without change or modification of the technical spirit or essential characteristics of the present disclosure. Therefore, it should be understood that the aforementioned examples are only provided by way of example and not provided to limit the present disclosure.

1. A method of treating colon cancer, the method comprising:
administering extracellular vesicles loaded with an MACC1 inhibitor or a composition comprising the extracellular vesicles as an active ingredient to a subject in need thereof.

2. The method according to claim 1, wherein the extracellular vesicles comprise a TM4SF5-targeting peptide.

3. The method according to claim 2, wherein the peptide comprises the amino acid sequence of SEQ ID NO: 1.

4. The method according to claim 1, wherein the inhibitor is one selected from the group consisting of miRNA, short hairpin RNA (shRNA), small interference RNA (siRNA), ribozyme, DNAzyme, PNA, an antibody, and an aptamer.

5. The method according to claim 4, wherein the inhibitor is miR143.

6. The method according to claim 5, wherein the miR143 comprises the nucleotide sequence SEQ ID NO: 3.

7. The method according to claim 1, wherein the extracellular vesicles are derived from adipose-derived stem cells.

8. The method according to claim 1, wherein the composition is administered intravenously.

9. The method according to claim 1, wherein the composition is pharmaceutical composition.


SEQUENCE LISTING (3 sequences)

SEQ ID NO: 1 - protein, 27 aa, synthetic construct
TAGAYLLNRTLWDRCEAPPRVVPWNVT

SEQ ID NO: 2 - DNA, 81 bp, synthetic construct
  source 1..81: mol_type = other DNA
acagcaggagcatacttgttgaatagaaccctgtgggaccgatgcgaagctccaccccgagtagttccgtggaatgttact

SEQ ID NO: 3 - RNA, 22 nt, synthetic construct
  source 1..22: mol_type = other RNA
ggtgcagtgctgcatctctggt